(19) United States
(12) Patent Application Publication
(10) Pub. No.: US 2005/0055067 A1
(43) Pub. Date: Mar. 10, 2005

(54) Medium and low frequency electro-stimulated massaging pants

(76) Inventor: Tsung-I Yu, Taipei (TW)

Correspondence Address: BACON & THOMAS, PLLC, 625 SLATERS LANE, FOURTH FLOOR, ALEXANDRIA, VA, 22314, US

(21) Appl. No.: 10/658,256

(22) Filed: Sep. 10, 2003

(51) Int. Cl. A61N 1/18

(52) U.S. Cl. 607/48

(57) ABSTRACT

A pair of medium and low frequency electro-stimulated massaging pants embeds a plurality of conductive strips on predetermined locations therein. Each of the conductive strips has a first end connected with at least one conductive plate and a second end connected with a conductive button. The conductive plates are in direct contact with the human body as the pants are put on. The conductive buttons are clustered on a predetermined location of the pants, which can be coupled with a corresponding set of conductive buttons on an electro-stimulating controller so as to mount the controller onto the pants. The electro-stimulating controller charges the conductive plates to produce the effect of a medium and low frequency electro-stimulated massage.

### BACKGROUND OF THE INVENTION

(a) Field of the Invention

The present invention relates to medium and low frequency electro-stimulated massaging devices, and more particularly to a pair of medium and low frequency electro-stimulated massaging pants, which is provided with a clustered conductive button set for mounting an electro-stimulating controller thereon so as to achieve the effect of massage or fat reduction.

(b) Description of the Prior Art

The devices for medium and low frequency wave massaging in the prior art are mainly composed of a controller and electrodes made of silica gel (or conducting gel or electrode cups made of silica gel). Those devices are disadvantageous in high production cost and low electrical conductivity and can only be applied in special occasions. Especially, silica gel must be smeared over body portions the electro-stimulated massage is applied to, which inevitably causes discomfort and cleansing problem to a user. Furthermore, silica-gel plates and silica-gel cups are both hard objects, which are heavy and uncomfortable to wear, substantially reducing the portability thereof.

### SUMMARY OF THE INVENTION

The primary objective of the present invention is to provide a pair of medium and low frequency electro-stimulated massaging pants having a plurality of conductive strips, which are made of electrically conductive cloths. Each of the conductive strips has a first end connected with a conductive plate and a second end connected with a conductive button. The conductive plates are in direct contact with the human body when the pants are put on. The conductive buttons are clustered on a predetermined location of the pants, which can be coupled with a corresponding set of conductive buttons on an electro-stimulating controller so as to mount the controller onto the pants. The electro-stimulating controller charges the conductive plates to provide the effect of a medium and low frequency electro-stimulated massage, which may actuate a rehabilitating exercise, promote blood circulation and induce fat reduction.

The various objects and advantages of the present invention will be more readily understood from the following detailed description when read in conjunction with the appended drawings.

### BRIEF DESCRIPTION OF THE DRAWINGS

FIG. 1 is a front view of the present invention with the controller separated from the pants.

FIG. 2 is a front view of the present invention with the controller connected with the pants.

FIG. 3 is a front perspective view of the electro-stimulating controller of the present invention.

FIG. 4 is a rear perspective view of the electro-stimulating controller of the present invention.

FIG. 5 is a cross-sectional side view of the electro-stimulating controller mounted on the pants.

FIG. 6 shows the connectivity of the central integrated circuit of the controller of the present invention.

FIG. 7 is the circuit layout of the controller of the present invention.

FIG. 8 is an exploded perspective view of the conductive strips being guided to the conductive buttons.

FIG. 9 is an exploded view of the inducing terminal of a conductive strip and a conductive plate before they are sewn together.

FIG. 10 is an exploded view of the inducing terminal of a conductive strip and a conductive plate after they are sewn together.

FIG. 11 is a perspective view of a connecting means by which a conductive plate is adhesively connected to a conductive strip with adhesive pieces.

FIG. 12 illustrates a preferred embodiment of the present invention wherein the conductive strips are being sewn with the elastic waistband.

FIG. 13 illustrates the preferred embodiment of the present invention in FIG. 12 wherein the conductive strips are sewn with the elastic waistband.

FIG. 14 illustrates a preferred embodiment of the present invention wherein the pants are toreador pants.

FIG. 15 illustrates a preferred embodiment of the present invention wherein the pants are tight underpants.

FIG. 16 illustrates a preferred embodiment of the present invention wherein a wire set connects the conductive buttons on the pants and another set of conductive buttons.

FIG. 17-FIG. 20 illustrate preferred embodiments of the present invention wherein the conductive plates on various types of pants are deployed corresponding to various body portions.

FIG. 21 shows the conductive buttons at the extended end of a conductive strip being covered by overlapped two-piece cloth covers.

### DETAILED DESCRIPTION OF THE PREFERRED EMBODIMENTS

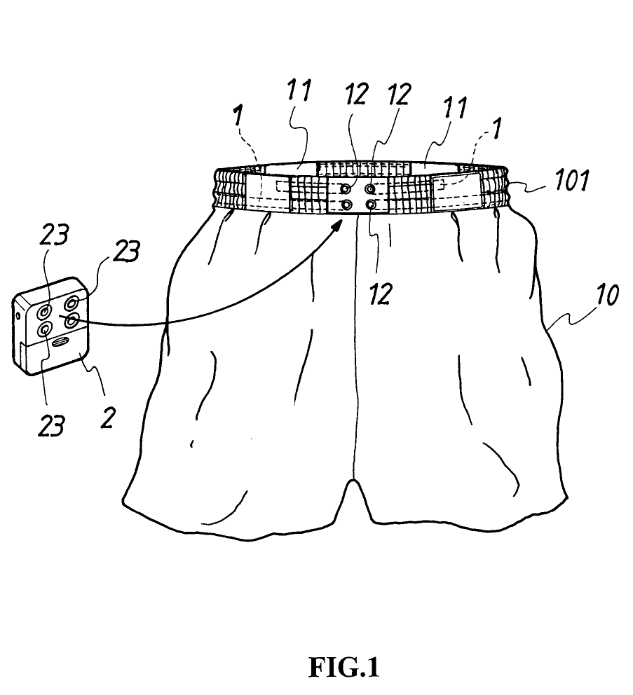

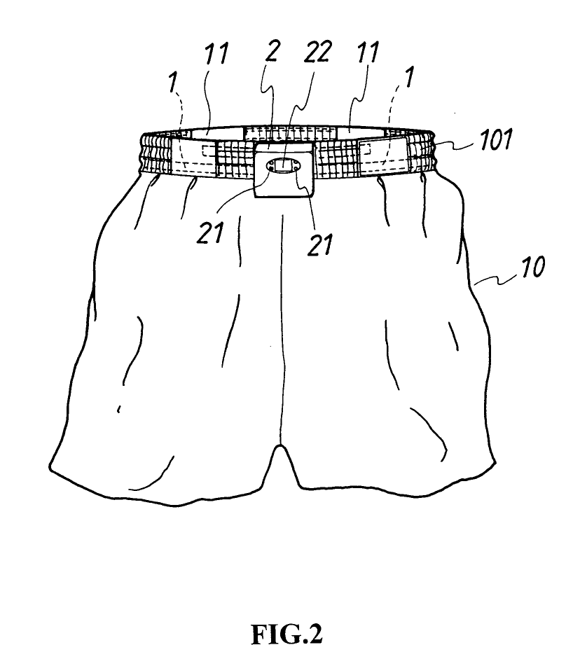

Referring to FIG. 1 and FIG. 2, a pair of medium and low frequency electro-stimulated massaging pants according to the present invention is basically a pair of pants 10 having a plurality of conductive strips 1 attached on selective locations thereon. The conductive strips 1 are installed along an elastic band 101 and/or on other predetermined locations of the pants 10. Each of the conductive strips 1 has a first end connected with a conductive plate 11 and a second end connected with a conductive button 12. The conductive buttons 12 are clustered on a pre-determined location of the pants 10, which can be coupled with a corresponding set of conductive buttons 23 on an electro-stimulating controller 2 so as to mount the controller 2 onto the pants 10, as shown in FIG. 2. The electro-stimulating controller 2 charges the conductive plates 11, which are in direct contact with the human body when the pants 10 are put thereon, to produce the effect of an medium and low frequency electro-stimulated massage.

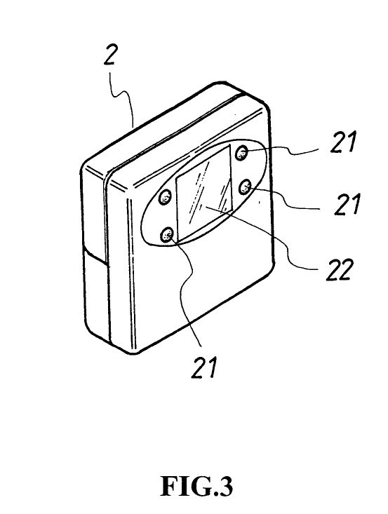

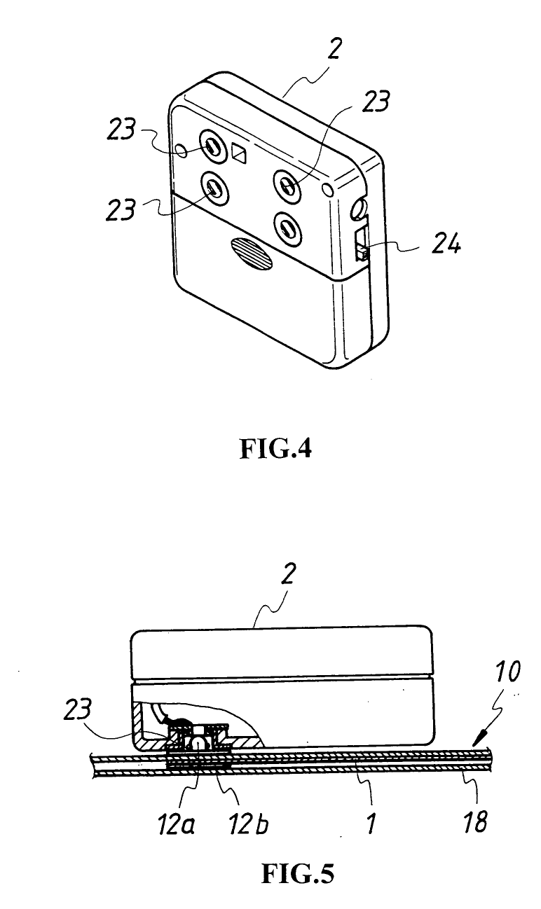

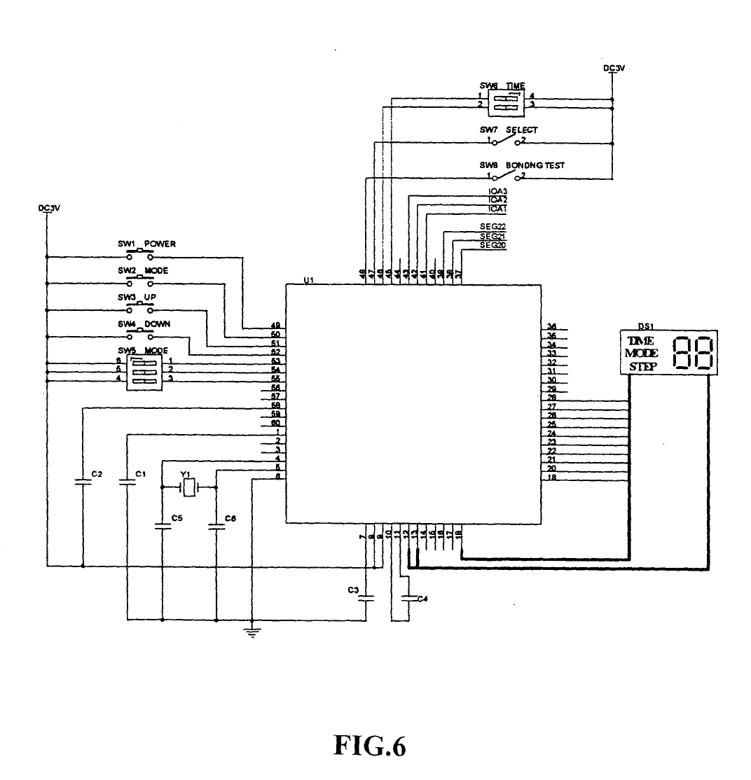

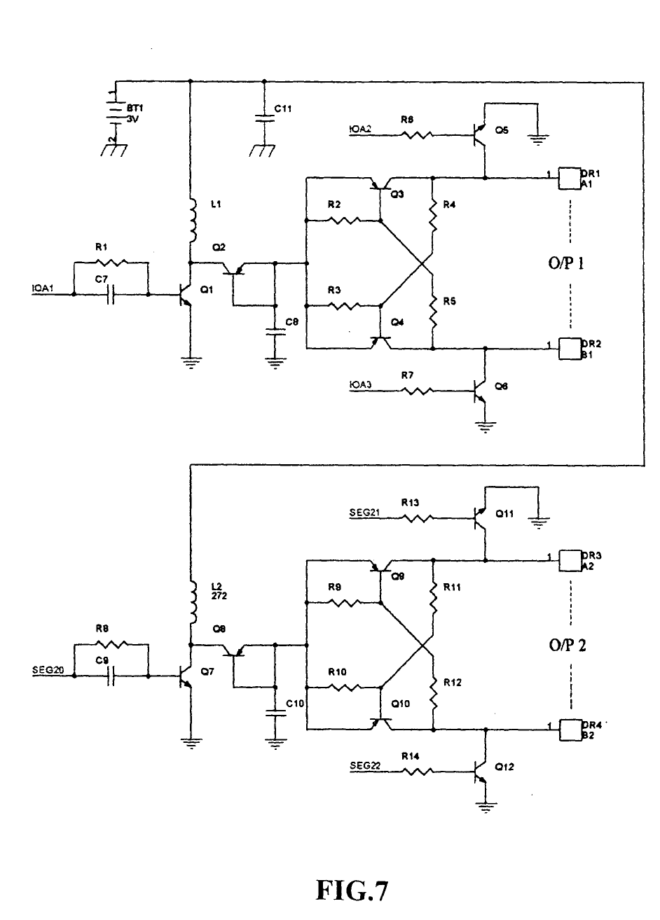

Referring to FIG. 3 to FIG. 7, the electro-stimulating controller 2 is provided with a central integrated circuit (IC) and a charging/discharging circuit. The IC transports a pulsed-wave signal to the circuit for controlling the charging/discharging of the capacitors and inductors thereon from which a high voltage is generated for providing an electro-stimulating effect. The IC further adjusts the bandwidth of the pulsed-wave signal, in a range from 1 Hz to 150 Hz, to produce massaging effect of various strengths. Furthermore, the electro-stimulating controller 2 has a plurality of control buttons 21 for respectively selecting current up/down, operation time, power on/off and massage mode. The electro-stimulating controller 2 further contains an LCD display for displaying the operation status. The pulsed high voltage generated by the internal circuit (as shown in FIG. 5 and FIG. 6) is connected to a plurality of conductive buttons 23, which then form an output terminal. A stepping switch 24 is installed on a lateral side of the electro-stimulating controller 2 for selecting the charging region.

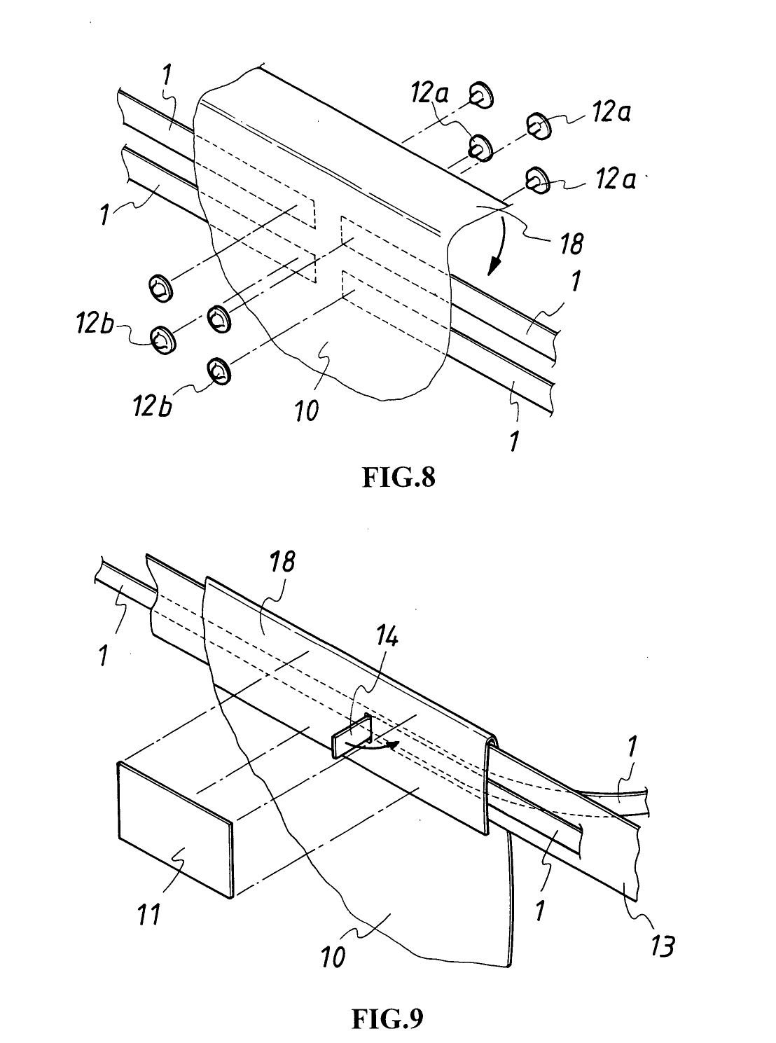

Referring to FIG. 8, each of the above-mentioned conductive strips 1 is secured in the elastic band 101 of the pants 10 and has the second end connected to a conductive button 12, which consists of a female piece 12a and a male piece 12b, respectively attached on each side of the elastic band 101. As the female piece 12a and the male piece 12b are being rivet-connected, the associated conductive strip 1 is secured within the elastic band 101, whereas the female piece 12a extends outside the pants 10, providing a connection to a conductive button 23 on the electro-stimulating controller 2, as shown in FIG. 2 or FIG. 5.

Referring to FIG. 9, the conductive strips 1 of the present invention can each be connected with more conductive plates 11. The conductive strips 1 are arranged either in parallel separated by a proper spacing or in an overlapped fashion and insulated by an insulating material 13, as shown in FIG. 9. The inducing terminal 14 of a conductive strip 1 is extended out of the elastic band 101 for touching a conductive plate 11 as the plate 11 is sewn on to the pants 10.

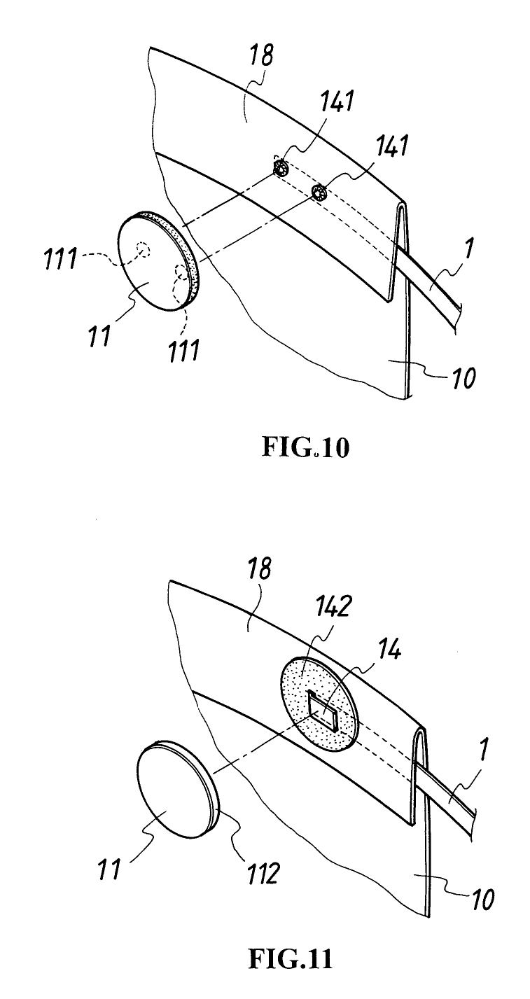

As shown in FIG. 10, another preferred embodiment of the present invention has a different means of connecting a conductive plate 11 and the inducing terminal 14 of a conductive strip 1. The inducing terminal 14 is provided with at least one conductive button 141, and the conductive plate is provided with at least one corresponding conductive button 111. The conductive plate 11 is mounted onto the inducing terminal 14 by riveting the conductive button 111 and the conductive button 141 together, which makes the conductive plate 11 detachable.

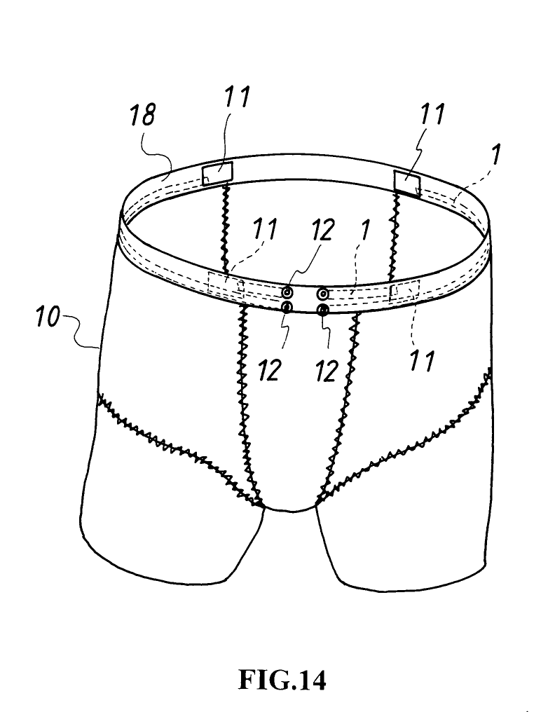

As shown in FIG. 14, another preferred embodiment of the present invention has an inducing terminal 14 that is provided with an adhesive patch 142 and a conductive plate 11 that is provided with a corresponding adhesive patch 112. The conductive plate 11 is mounted onto the inducing terminal 14 by sticking the adhesive patch 112 and the adhesive patch 142 together.

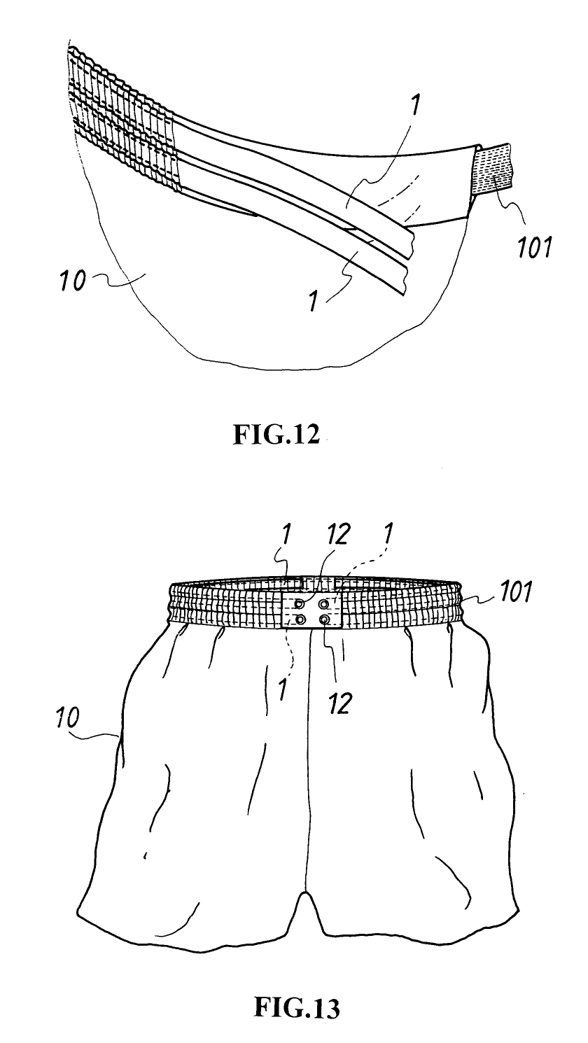

As shown in FIG. 12 and FIG. 13, another preferred embodiment of the present invention has a plurality of conductive strips 1 sewn into the elastic band 101 and having the same wrinkled form as the elastic band 101.

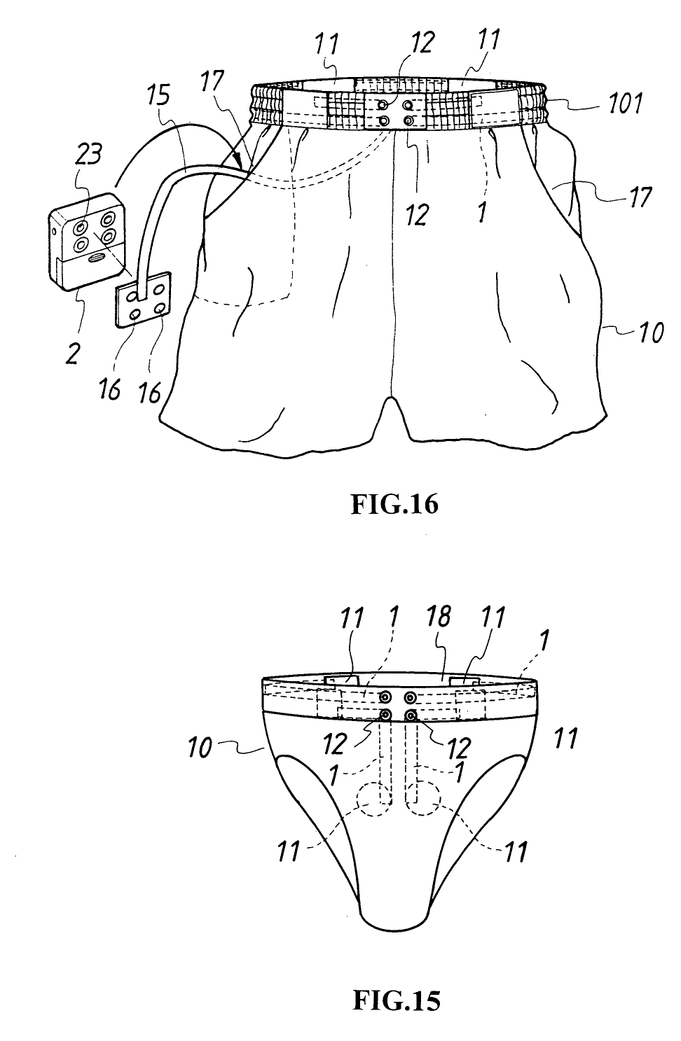

The pants 10 according to the present invention need not to have a fixed form. It can be selected from male underpants, female underpants, outer pants and toreador pants. The preferred embodiment shown in FIG. 14 is the present invention applied to a pair of toreador pants, whereas the preferred embodiment shown in FIG. 15 is the present invention applied to a pair of ordinary underpants. When embodied in toreador pants or other tight underpants, more conductive plates can be added on locations corresponding to other body portions, which are connected to conductive strips 1 extended from the elastic band 101.

Referring to FIG. 16, the medium and low frequency electro-stimulated massaging pants according to the present invention may have the electro-stimulating controller 2 separable from the pants 10. A set of wires 15 is used to connect the conductive buttons 12 of the pants 10. The free end of the wire set 15, extending from a pocket 17, is provided with a set of conductive buttons 16. The conductive buttons 16 are capable of being coupled with the conductive buttons 23 on the electro-stimulating controller 2 so that the electro-stimulating controller 2 can be put in the pocket 17.

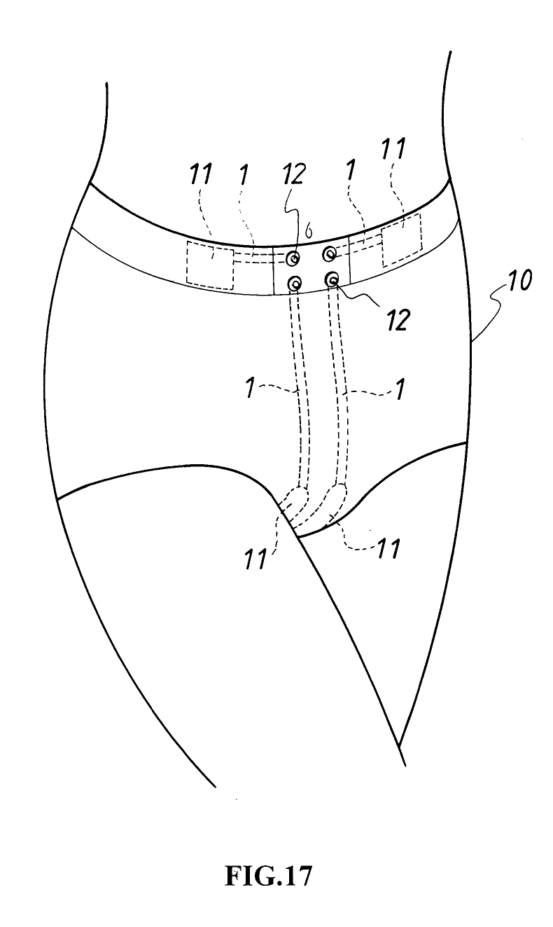

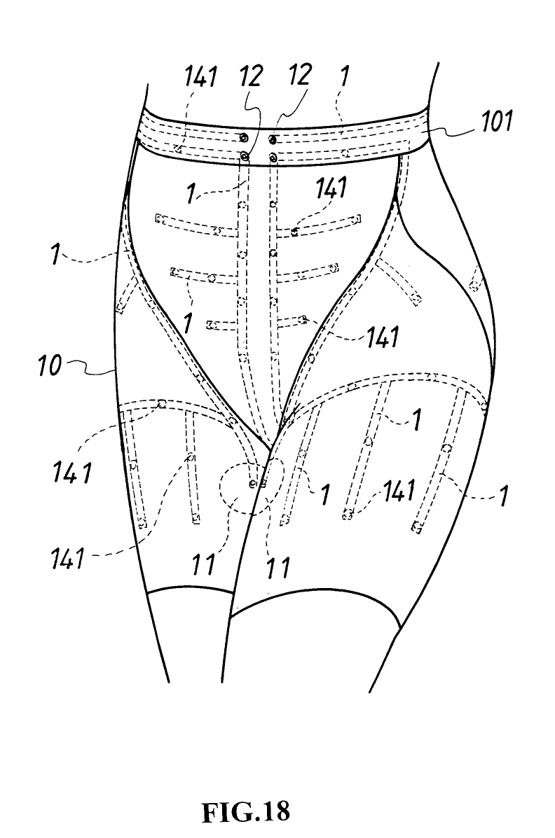

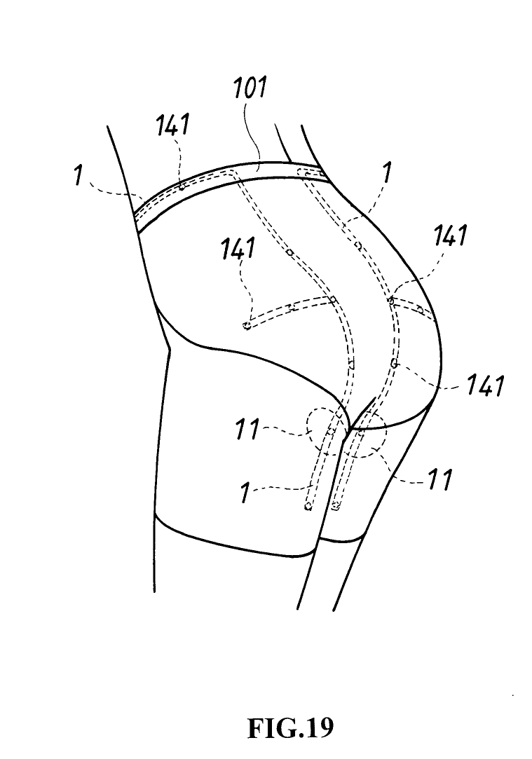

The above-mentioned pants 10, taking the forms of toreador pants or tight underpants, have closer contact with the human body, and therefore may have conductive strips 1 and conductive plates 11 deployed on various locations of the pants. Referring to FIG. 17 to FIG. 20, a variety of such preferred embodiments have conductive plates 11 connected in series or in parallel by a plurality of conductive strips 1 and installed on locations on the inner surface of the pants, which correspond to acupuncture points or fat-accumulating places. Alternatively, a selected place where a conductive strip 1 extends can be provided with a conductive button 141 for mounting a conductive plate 11 when an electro-stimulated massage is designated to the place. In addition to the effects of promoting circulation and fat reduction, the electro-stimulated massage applied to some sensitive body portions would produce a sexually provoking effect.

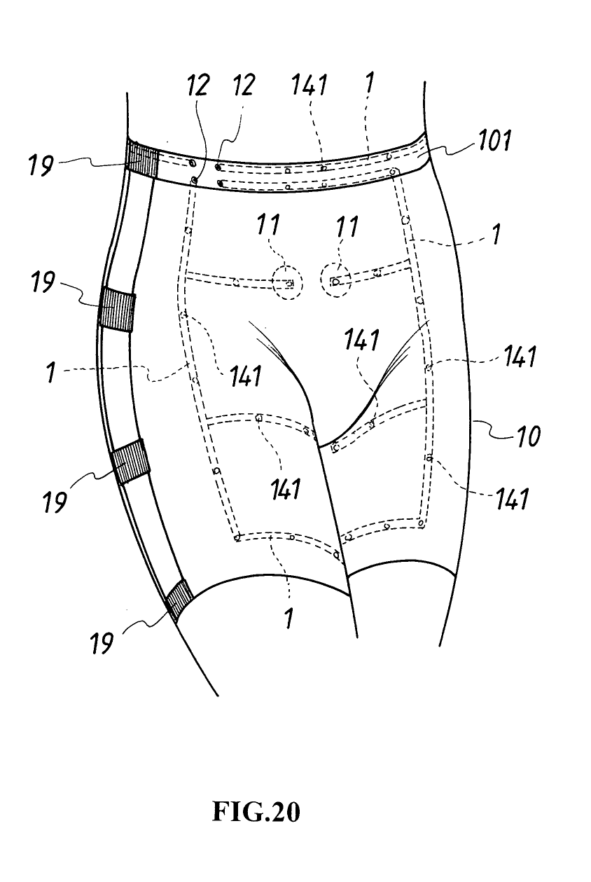

Referring to FIG. 20, a plurality of elastic bands 19 are added on one or two lateral sides of the pants 10 for accommodating users of various sizes.

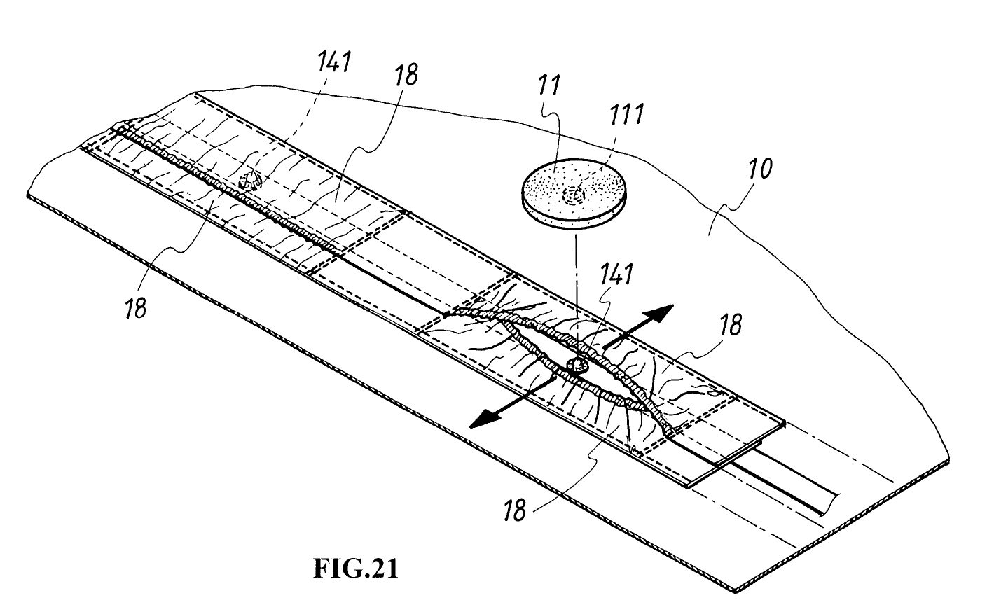

Referring to FIG. 21, the above-mentioned conductive buttons 141 on the pants 10 are each covered by a cloth cover 18 formed by two overlapped pieces, and the conductive buttons 141 are thereby electrically insolated. To use a conductive button 141, two pieces of a cloth cover 4 are pushed aside to enable a connection with a conductive plate 11 of a conductive strip 1. The cloth cover 18 can also be of one piece, which is lifted when the conductive button 141 underneath is to be used.

The conductive strips 1 of the present invention are made of ordinary electro-conductive materials, and preferably soft electro-conductive cloths.

Although the present invention has been described with reference to the preferred embodiments, it will be understood that the invention is not limited to the details described thereof. Various substitutions and modifications have been suggested in the foregoing description, and others will occur to those of ordinary skill in the art. Therefore, all such substitutions and modifications are intended to be embraced within the scope of the invention as defined in the appended claims.

1. A pair of medium and low frequency electro-stimulated massaging pants comprising:
a pair of pants;
a plurality of conductive plates attached on an inner surface of said pants and in direct contact with a human chest when said pants are put on;
a plurality of conductive buttons clustered on a predetermined location of said pants;
a plurality of conductive strips, each having a first end connected to one of said conductive plates and a second end connected to one of said conductive buttons; and
an electro-stimulating controller having a corresponding set of conductive buttons for coupling with said conductive buttons of said pants, said electro-stimulating controller charging said conductive plates to produce an effect of medium and low frequency electro-stimulated massage.

2. The medium and low frequency electro-stimulated massaging pants of claim 1, wherein said conductive plates are connected to a positive electrode and a negative electrode of said electro-stimulating controller.

3. The medium and low frequency electro-stimulated massaging pants of claim 1, wherein said conductive strips are embedded within an elastic waistband.

4. The medium and low frequency electro-stimulated massaging pants of claim 1, wherein said electro-stimulating controller is provided with a central integrated circuit (IC); said IC is used for transporting a pulsed-wave signal to control the charging/discharging of a group of capacitors and inductors for generating a high voltage; said IC further adjusts the bandwidth of said pulsed-wave signal so as to generate high voltages for providing medium and low frequency electro-stimulated massaging effects of various strengths.

5. The medium and low frequency electro-stimulated massaging pants of claim 4, wherein said bandwidth of said pulsed-wave signal is selected in a range from 1 Hz to 150 Hz.

6. The medium and low frequency electro-stimulated massaging pants of claim 1, wherein said pulsed high voltage generated by said electro-stimulating controller is guided to a plurality of conductive buttons thereon; said conductive buttons of said electro-stimulating controller then form an output terminal.

7. The medium and low frequency electro-stimulated massaging pants of claim 1, wherein a stepping switch is installed on a lateral side of said electro-stimulating controller for selecting a charging region.

8. The medium and low frequency electro-stimulated massaging pants of claim 1, wherein said conductive strips are in a parallel arrangement separated by a proper spacing.

9. The medium and low frequency electro-stimulated massaging pants of claim 1, wherein said conductive strips are arranged in an overlapped fashion and insulated by an insulating material; an inducing terminal of each of said conductive strips is extended out of said elastic waistband; said conductive plates are sewn on said pants to connect with said inducing terminals.

10. The medium and low frequency electro-stimulated massaging pants of claim 1, wherein an inducing terminal of each of said conductive strips is provided with at least one conductive button, and each of said conductive plates is provided with at least one corresponding conductive button; said conductive plates are mounted onto said inducing terminals by riveting said pairs of conductive buttons together.

11. The medium and low frequency electro-stimulated massaging pants of claim 1, wherein an inducing terminal of each of said conductive strips is provided with an adhesive patch, and each of said conductive plates is provided with a corresponding adhesive patch; said conductive plates are mounted onto said inducing terminals by sticking said pairs of adhesive patches together.

12. The medium and low frequency electro-stimulated massaging pants of claim 1, wherein said conductive strips are sewn on an inner surface of said pants.

13. The medium and low frequency electro-stimulated massaging pants of claim 1, wherein said pants are toreador pants or tight underpants; said conductive strips and said conductive plates are distributed on locations of said pants corresponding to various body portions.

14. The medium and low frequency electro-stimulated massaging pants of claim 1, wherein a set of wires is connected with said conductive buttons clustered on said pants; a free end of said wire set is provided with a set of conductive buttons capable of being mounted onto said electro-stimulating controller.

15. The medium and low frequency electro-stimulated massaging pants of claim 1, wherein said conductive strips, connected in series or in parallel, extend to various portions of the human body where said conductive plate or a predetermined number of conductive buttons are connected.

16. The medium and low frequency electro-stimulated massaging pants of claim 1, wherein said pants are toreador pants or tight underpants; a plurality of auxiliary elastic bands are installed on one or two lateral sides of said pants.

17. The medium and low frequency electro-stimulated massaging pants of claim 1, wherein said conductive buttons at said extended ends of said conductive strips are each covered by an insulating cloth cover that is formed by either two overlapped pieces of cloth or a single piece of cloth.

18. The medium and low frequency electro-stimulated massaging pants of claim 1, wherein said conductive strips are made of electrically conductive cloths.
